(19) United States
(12) Patent Application Publication
(10) Pub. No.: US 2014/0276231 A1
(43) Pub. Date: Sep. 18, 2014

(54) Graft Cartilage Management Systems and Methods

(71) Applicant: Thomas E. Wood, Guilford, CT (US)

(72) Inventor: Thomas E. Wood, Guilford, CT (US)

(73) Assignee: Accelerated Orthopedic Technologies, LLC, Guilford, CT (US)

(21) Appl. No.: 13/832,108

(22) Filed: Mar. 15, 2013

(51) Int. Cl. A61B 17/3205; A61F 2/46; A61B 5/00

(52) U.S. Cl. CPC A61B 17/3205; A61B 5/4514; A61B 5/4528; A61F 2/4657
USPC 600/587; 606/170

(57) ABSTRACT

Exemplary embodiments are directed to systems and methods of graft cartilage management for repairing a defect in a joint of a patient that include providing a donor database that includes information on a plurality of donor sites, receiving first data relating to the defect of the joint of the patient, and identifying, based on the first data, at least one donor site from the donor database for harvesting a graft specimen dimensioned greater than the defect in the joint of the patient. Exemplary embodiments are also directed to systems and methods of graft cartilage management that include a donor database of like and unlike joint donor sites and identifying at least one unlike joint donor site for harvesting a graft. In addition, exemplary embodiments are directed to systems and methods of defect region management that include a database of joint defects and identifying at least one joint defect for repair.

### TECHNICAL FIELD

The present disclosure relates to instruments, methods and systems for use in harvesting graft materials, e.g., cartilage, from donor sites. More particularly, the present disclosure provides apparatus and systems that may be used by clinicians to acquire osteochondral grafts of desired shapes, sizes and/or depths in an efficient and reliable manner, and to implant such grafts in desired locations. The disclosed instruments, methods and systems have wide clinical utility and applicability, and may be employed with beneficial results to harvest and/or implant allograft, autograft and/or synthetic materials.

### BACKGROUND

Articular cartilage is a complex structure that, once damaged, has little capacity for permanent repair. One technique that has received attention for addressing cartilage-related issues involves repair with living hyaline cartilage through osteochondral autograft transplant. The procedure is known as mosaicplasty and generally involves removing injured tissue from a damaged area. One or more cylindrical sockets are drilled into the underlying bone and a cylindrical plug graft—consisting of healthy cartilage from the knee—is implanted in each socket.

As discussed in PCT applications entitled “Systems, Devices and Methods for Cartilage and Bone Grafting” and “Instruments, Methods and Systems for Harvesting and Implanting Cartilage Materials,” which published as WO 2009/154691 A9 (corrected version) and WO 2011/008968 A1, respectively, commercially available instruments for use in mosaicplasty procedures include Acufex instruments available from Smith & Nephew, Inc. (Andover, Mass.), the COR System available from Innovasive Technologies (Marlborough, Mass.), and the Arthrex Osteochondral Autograft Transfer System available from Arthrex (Naples, Fla.). The contents of the foregoing PCT applications are incorporated herein by reference.

Despite efforts to date, a need remains for instruments and systems for efficient, effective and reliable access to desired graft/cartilage sites and removal of desired graft/cartilage tissue. In addition, a need remains for instruments/systems that facilitate graft/cartilage access and/or removal in a minimally invasive manner. Still further, a need remains for instruments/systems that facilitate effective, efficient and reliable selection of donor graft/cartilage sites and/or graft/cartilage source materials that geometrically match the removed cartilage tissue and/or void region. These and other needs are met by the instruments/systems and associated methods disclosed herein.

### SUMMARY

In accordance with embodiments of the present disclosure, an instrument is provided for capturing a surface topography of a defect region. In particular, the exemplary topographical instrument generally includes a plurality of movably mounted elongated rod members and a locking mechanism for releasably securing the plurality of elongated rod members relative to each other and relative to the instrument axis. The plurality of elongated rod members may be advantageously configured and/or oriented to capture an entire surface topography of the anatomical location of a defect region, including a combination of a peripheral surface topography and a central surface topography. Further, the plurality of elongated rod members are generally independently translatable relative to each other (and relative to the instrument axis) in order to capture an accurate surface topography of the defect region.

In accordance with another embodiment of the present disclosure, a device that includes graft harvesting functionality is provided, generally including an elongated shaft and a detachable cutting member mounted with respect to the elongated shaft and operative to form a cavity or void region. In exemplary embodiments of the present disclosure, the disclosed device is adapted to form a cavity/void region of a predetermined geometry and/or depth. The exemplary device may further include a plurality of elongated rod members for capturing a topography, e.g., a peripheral surface topography, of the anatomical location in proximity to the intended or actual location of the cavity/void region, a broach member that may advantageously include structural feature(s) for at least one of cleaning and smoothing the periphery of the cavity/void region, and a hammer mechanism configured to slide relative to the axis of the shaft.

In accordance with another embodiment of the present disclosure, an instrument for removing material from a defect region is provided. The exemplary instrument generally includes a template configured and dimensioned to receive a mounting track. The exemplary instrument generally further includes a cutter configured and dimensioned to be inserted into the template. In particular, the cutter can include a travel indication feature for indicating a cutter position within the template. The template can include a peripheral template track for receiving placement of the mounting track, which further facilitates placement and anchoring of the template relative to an anatomical structure. The travel indication feature can be, e.g., a bushing, and the alignment of a template outer periphery with a travel indication feature outer periphery can indicate the cutter position within the template.

In accordance with yet another embodiment of the present disclosure, a method for defect repair is provided, generally including the steps of establishing a referential orientation of an instrument relative to an anatomical location, capturing a surface topography of the anatomical location of the defect region (e.g., a complete/entire surface topography), forming a defect region cavity that defines a cavity region geometry in the anatomical location, and using the captured surface topography of the anatomical location of the defect region to identify a donor location and/or graft source with a complementary surface topography as a harvest region or source of graft material for a plug to fill the defect region cavity. The cavity region geometry may be predefined according to the disclosed method. The graft material may be an allograft, autograft and/or synthetic material.

According to exemplary embodiments of the disclosed method, the defect region cavity is generally formed with a predefined depth and is formed at substantially a right angle relative to the axis of the instrument used to form such defect region cavity. The exemplary method generally further includes using a detachable broach member for cleaning and/or smoothing a peripheral wall associated with the defect region cavity and using a plurality of elongated rod members for capturing a surface topography of the anatomical location in proximity to the defect region cavity. Further still, the exemplary method generally includes using a cutter to obtain a graft plug from a harvest region or source of graft material (e.g., autograft, allograft and/or synthetic material), using a cutter guide to trim the graft plug to a predefined depth, using an axially movable member (e.g., a structure that also functions as the broach member) to eject the graft plug from the cutter, and introducing the graft plug into the defect region cavity. In general, the defect region cavity may be advantageously formed using a template having a predefined opening geometry. The graft plug is typically obtained using a cutter that defines a cutting geometry in which the predefined opening geometry of the template and the cutting geometry of the cutter correspond (or substantially correspond) to each other.

In accordance with yet another embodiment of the present disclosure, an exemplary method of graft cartilage management for repairing a defect in a joint of a patient is provided that includes providing a donor database which includes information on a plurality of donor sites. The method includes receiving first data relating to the defect of the joint of the patient, e.g., at least one of imaging data of the defect, imaging data of an area surrounding the defect, a size of the defect, a surface topography of the defect, a joint anatomical characteristic, and the like. The joint anatomical characteristic can be at least one of, e.g., a cartilage density, a cartilage thickness, a cartilage resiliency, and the like. The method further includes identifying, based on the first data, at least one donor site from the donor database for harvesting a graft specimen dimensioned greater than the defect in the joint of the patient.

The exemplary method includes prioritizing the identified at least one donor site based on, e.g., a donor site availability, a donor site compatibility, and the like. The identified at least one donor site generally includes a like joint and/or an unlike joint with respect to the joint of the patient. The method further includes harvesting the graft specimen from the at least one donor site and delivering the graft specimen to a surgeon. Second data relating to the defect of the joint of the patient is then collected. The second data generally relates to at least one of, e.g., imaging data of the defect, imaging data of an area surrounding the defect, a size of the defect, a surface topography of the defect, a joint anatomical characteristic, and the like. Further, the method includes harvesting a graft, i.e., a custom graft for the defect, from the graft specimen based on the second data relating to the defect of the joint of the patient.

In accordance with yet another embodiment of the present disclosure, an exemplary graft cartilage management system for repairing a defect in a joint of a patient is provided that includes a donor database which includes information on a plurality of donor sites. The system further includes a processing device configured to access the donor database and receive first data relating to the defect of the joint of the patient. The processing device can be further configured to identify, based on the first data, at least one donor site from the donor database for harvesting a graft specimen dimensioned greater than the defect in the joint of the patient.

The system generally includes an apparatus, e.g., an imaging and/or non-imaging apparatus, to capture the first data relating to the defect of the joint of the patient. The first data can be at least one of, e.g., imaging data of the defect, imaging data of the area surrounding the defect, a defect size, a defect surface topography, a joint anatomical characteristic, and the like. The system generally includes a harvesting device for harvesting the graft specimen from the at least one donor site. Further, the system includes an apparatus to capture second data relating to the defect of the joint of the patient and a harvesting device for harvesting a graft from the graft specimen based on the second data relating to the defect of the joint of the patient.

In accordance with yet another embodiment of the present disclosure, an exemplary method of graft cartilage management for repairing a defect in a joint of a patient is provided that includes providing a donor database. The donor database includes information on a plurality of donor sites which include like joint donor sites and unlike joint donor sites relative to the joint of the patient. The method generally includes receiving first data relating to the defect of the joint of the patient. Further, the method generally includes identifying, based on the first data, at least one unlike joint donor site from the donor database for harvesting a graft to repair the defect in the joint of the patient. The donor database generally includes correlation information for determining which unlike joints are compatible with the defect in the joint of the patient.

The identified at least one unlike joint donor site can be compatible with the joint of the patient based on at least one of, e.g., imaging data of the defect, imaging data of an area surrounding the defect, a size of the defect, a surface topography of the defect, a joint anatomical characteristic of the defect, and the like. The joint anatomical characteristic can be at least one of, e.g., a cartilage density, a cartilage thickness, a cartilage resiliency, and the like.

In accordance with yet another embodiment of the present disclosure, an exemplary graft cartilage management system for repairing a defect in a joint of a patient is provided that includes a donor database including information on a plurality of donor sites. The plurality of donor sites include like joint donor sites and unlike joint donor sites relative to the joint of the patient. The system generally includes a processing device configured to access the donor database and receive first data relating to the defect of the joint of the patient. The processing device is generally configured to identify, based on the first data, at least one unlike joint donor site from the donor database for harvesting a graft to repair the defect in the joint of the patient.

In accordance with yet another embodiment of the present disclosure, an exemplary method of defect region management for repairing a defect in a joint of a patient is provided that includes providing a database comprising information on a plurality of joint defects. The method generally includes receiving first data relating to a donor site and identifying, based on the first data, at least one joint defect from the database for repair by a graft harvested from the donor site.

The first data includes at least one of, e.g., imaging data of the donor site, a size of the donor site, a surface topography of the donor site, a joint anatomical characteristic, and the like. The method generally includes prioritizing the identified at least one joint defect and harvesting the graft from the donor site. The graft can be dimensioned greater than the identified at least one joint defect. Thus, the exemplary method can further include collecting second data relating to the identified at least one joint defect and harvesting a custom graft based on the second data relating to the identified at least one joint defect.

In accordance with yet another embodiment of the present disclosure, an exemplary defect region management system for repairing a defect in a joint of a patient is provided that includes a database which includes information on a plurality of joint defects. The system further includes a processing device configured to access the database and receive first data relating to a donor site. The processing device can be configured to identify, based on the first data, at least one joint defect from the database for repair by a graft harvested from the donor site.

Thus, the exemplary instruments, systems and methods described herein provide efficient, effective and reliable access to desired cartilage sites of like and/or unlike joints, removal of desired cartilage tissue and selection of donor cartilage sites or sources of graft material (allograft, autograft and/or synthetic) which geometrically match (or substantially match) an associated cavity region, and facilitate cartilage access and/or removal in a minimally invasive manner. The exemplary systems and methods described herein also provide efficient, effective and reliable selection and removal of donor cartilage sites or sources of graft material greater than the defect area to be repaired such that the graft material can be customized by a surgeon at the time of the operation. In addition, the exemplary systems and methods described herein provide efficient, effective and reliable selection of a joint defect site based on information received about a donor site.

Other objects, features, functions and benefits will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

FIG. 1 is a schematic view of an exemplary trial member according to an illustrative embodiment of the present disclosure;

FIG. 2 depicts an exemplary template according to an exemplary embodiment of the present disclosure;

FIG. 3 depicts an exemplary template assembly according to an exemplary embodiment of the present disclosure;

FIG. 4 depicts an exemplary template assembly and cutter according to the present disclosure;

FIG. 5 depicts an exemplary template assembly and cutter according to the present disclosure;

FIG. 6 is a schematic view of an exemplary graft harvesting device according to the present disclosure;

FIG. 7 depicts an exemplary cutting member and broach member of an exemplary graft harvesting device prior to insertion into a defect region cavity;

FIG. 8 depicts an exemplary graft harvesting device during insertion of an exemplary broach member into a defect region cavity;

FIG. 9 depicts an exemplary graft harvesting device after removal of an exemplary broach member from a defect region cavity;

FIG. 10 depicts an exemplary graft harvesting device prior to harvesting a donor graft;

FIG. 11 depicts an exemplary graft harvesting device post-harvesting a donor graft;

FIG. 12 depicts an exemplary cutter guide of an exemplary graft harvesting device for trimming a donor graft to a predefined depth;

FIG. 13 depicts an ejection of a donor graft from an exemplary graft harvesting device;

FIG. 14 is a schematic view of an alternative exemplary graft harvesting device according to an illustrative embodiment of the present disclosure;

FIGS. 15A and 15B are schematic views of an exemplary cutting member and broach member of an exemplary graft harvesting device according to illustrative embodiments of the present disclosure;

FIG. 16 is a flowchart of an exemplary method for defect repair according to the present disclosure;

FIG. 17 is a diagram of an exemplary surface mapping system according to the present disclosure;

FIG. 18 is a diagram of an exemplary computing system utilized with embodiments of the present disclosure;

FIG. 19 is a flowchart of an exemplary procedure to identify suitable sites for bone-cartilage grafts for repairing a defect region of a patient according to the present disclosure;

FIG. 20 is an exemplary arrangement of a system to acquire, process and store data according to the present disclosure;

FIG. 21 is a flowchart of an exemplary procedure of graft cartilage management according to the present disclosure;

FIG. 22 is a diagram of an exemplary surface mapping system according to the present disclosure; and

FIG. 23 is a flowchart of an exemplary procedure of defect region management according to the present disclosure.

### DESCRIPTION OF EXEMPLARY EMBODIMENTS

In accordance with embodiments of the present disclosure, an instrument is provided for capturing a surface topography of a defect region. In particular, the exemplary topographical instrument generally includes a plurality of elongated rod members and a locking mechanism for releasably securing the plurality of elongated rod members relative to each other and relative to the axis of the instrument. The plurality of elongated rod members may be configured and/or oriented to capture an entire surface topography of the anatomical location of a defect region, including a combination of a peripheral surface topography and a central surface topography. Further, the plurality of elongated rod members are generally independently translatable relative to each other in order to capture an accurate surface topography of the defect region.

In accordance with another embodiment of the present disclosure, a graft harvesting device is provided, generally including an elongated shaft and a detachable cutting member mounted with respect to the elongated shaft and operative to form a harvest cavity of a predetermined geometry. The detachable cutting member may advantageously be included in a detachable subassembly that delivers cutting functionality and potentially one or more additional functionalities. The exemplary device generally further includes a plurality of elongated rod members for capturing a surface topography of the anatomical location (e.g., a peripheral surface topography) in proximity to an existing or intended defect region cavity. The exemplary device may further include an axially movable broach member that includes structural feature(s) for cleaning and/or smoothing a peripheral wall associated with the defect region cavity, and a hammer mechanism configured to slide along the elongated shaft. The disclosed broach member may be advantageously included in the detachable subassembly that includes the cutting member, and may be axially movable relative to such cutting member.

In accordance with yet another embodiment of the present disclosure, an instrument for removing material from a defect region is provided. The exemplary instrument generally includes a template configured and dimensioned to receive a mounting track. The exemplary instrument generally further includes a cutter configured and dimensioned to be inserted into the template. In particular, the cutter can include a travel indication feature for indicating a cutter position within the template. The template can include a peripheral template track for receiving placement of the mounting track, which further facilitates placement and anchoring of the template relative to an anatomical structure. The travel indication feature can be, e.g., a bushing, and the alignment of a template outer periphery with a travel indication feature outer periphery can indicate the cutter position within the template.

With respect to FIG. 1, a schematic of an exemplary trial member 100 is presented according to an illustrative embodiment of the present disclosure. In particular, the exemplary trial member 100 generally includes a plurality of elongated rod members 101 that are movably mounted relative to each other and to the axis of the trial member. In the illustrated embodiment, the elongated rod members 101 are configured to capture the entire surface topography, e.g., a peripheral surface topography and a central surface topography, of a selected anatomical location. However, the present disclosure is not limited by or to such structural arrangement, and extends to structural arrangements where the plurality of elongated rods 101 do not cover the full extent of the peripheral and central surface topographies.

The plurality of elongated rod members 101 can be manufactured from a material suitable for medical purposes, e.g., stainless steel, titanium, cobalt or cobalt chrome, polymeric materials, and the like, and the selected anatomical location can be a defect region 104 of, e.g., cartilage 103 of a patient. The disclosed trial 100 may also be employed to capture surface topography at other locations, e.g., a donor site and/or an allograft or synthetic source of potential graft material. The plurality of elongated rod members 101 are further configured to be independently translatable relative to each other and can thereby capture the specific topography of the surface directly beneath the respective elongated rod members 101, permitting an accurate capture of the entire defect region 104 surface topography. It should be noted that the number of elongated rod members 101 depicted in FIG. 1 is for illustrative purposes only and that, e.g., a smaller elongated rod member 101 diameter can be utilized for greater accuracy, a larger elongated rod member 101 diameter can be utilized, a greater and/or smaller number of elongated rod members 101 can be utilized depending on the area of the defect region 104 or other region of interest, combinations thereof, and the like.

Still with reference to FIG. 1, the plurality of elongated rod members 101 are generally grouped together to provide a continuous and/or evenly distributed capture of the defect region 104 surface topography (or other region topography). Thus, in addition to capturing the peripheral surface topography of the defect region 104, the exemplary trial member 100 further captures the central, e.g., middle, surface topography of the defect region 104.

A locking member 102 can further be implemented for automatically securing the plurality of elongated rod members 101 relative to each other and relative to the axis of the device. The elongated rod members 101 are generally aligned in parallel, e.g., in aligned paths/conduits, to ensure accuracy of the captured surface topography. The locking member 102 can be configured as, e.g., a rubber O-ring, an elastic band, a sheet of silicone with a plurality of predefined openings/apertures positioned to accommodate passage of the elongated rod members 101 therethrough, a mechanical lock, and the like. As would be apparent to those of ordinary skill in the art, the locking member 102 generally provides radial resistance, e.g., a friction fit, to releasably lock the elongated rod members 101 in place in order to accurately capture the surface topography of the defect region 104.

In particular, while the trial member 100 is being lowered in proximity to defect region 104 in order to capture the surface topography, the elongated rod members 101 are free to independently translate along a vertical axis running the length of each elongated rod member 101 relative to each other and the locking member 102. Once the trial member 100 has been situated in an acceptable position, the locking member 102 can be actuated and/or automatically releasably locks the elongated rod members 101 in a configuration representative of the defect region 104 surface topography. Absent a mechanical lock, the locking force applied by an O-ring, elastic band or silicone sheet may be overcome by applying an adequate force on rod-by-rod basis.

Once the trial member 100 has been used to capture surface topography of a defect region 104 as described herein, the trial member 100 can then be implemented for identifying an allograft and/or autograft donor location or synthetic material as a harvest region for a plug based on a complementary surface topography, e.g., matching the defect region 104 surface topography to the surface topography of a donor location. The donor location may be, e.g., a joint of the patient, an allograft joint, or a xenograft material. Alternatively, the trial member 100 may be used to contour a synthetic material.

Turning now to FIG. 2, an exemplary template 201 is illustrated according to an exemplary embodiment of the present disclosure. In particular, the exemplary template 201 can be manufactured from, e.g., stainless steel, and can be utilized for forming a defect region cavity, e.g., removing material from a defect region 104 of the patient, and further generally includes a preformed geometric shape opening 202 of a desired and predefined shape. A template bottom portion 203 is adapted for placing the template 201 onto a desired location, e.g., the cartilage 103 of the patient. Thus, when the template 201 is placed onto the cartilage 103 of the patient, the preformed geometric shape opening 202 of the template 201 is configured and dimensioned to define the outer periphery of the defect region cavity to be formed. Although illustrated as substantially oval in shape, it should be understood that the preformed geometric shape opening 202 can be configured and dimensioned as circular and/or non-circular shapes depending on the shape of the defect region to be removed.

As depicted in FIG. 2, a cutter 204 generally includes a drill bit 205 and a stop element 206, e.g., a bushing, and can be utilized to form a defect region cavity by removing materials at defect region 104. The upper shaft of the cutter 204 is adapted for engagement with a drive mechanism (not pictured), as is readily apparent to persons skilled in the art. The drill bit 205 can be, e.g., a downcutting drill bit as discussed in a U.S. patent application entitled “Orthopedic Downcutting Instrument and Associated Systems and Methods,” which published as US 2011/0238070 A1. The contents of the foregoing U.S. patent application are incorporated herein by reference. The stop element 206 can abut the top surface of the template 201 to ensure a continuous and/or even depth of the defect region cavity being formed by providing support for the cutter 204 and preventing the drill bit 205 from penetrating deeper than the desired depth. The inner side surface of the preformed geometric shape opening 202 can further assist the user by guiding the drill bit 205. Of note, the stop element 206 may be sized such that its outer periphery substantially aligns with the outer periphery of the top surface of the template 201 when the drill bit 205 is in abutment (or substantial abutment) with an inner wall of the preformed geometric shape opening 202. In this way, a system user can determine when the drill bit 205 has reached its “outer” travel limit based on abutment with the inner wall of the preformed geometric shape opening 202 without visualization thereof. Accordingly, in such exemplary embodiments, when the side surface of the disk shaped stop element 206 and the template side surface 207 are aligned (or substantially aligned), it should be understood that the drill bit 205 has reached the inner surface of the preformed geometric shape opening 202. It should further be understood that the outer periphery of the top surface of the template 201 can be configured and dimensioned to match the geometry of the preformed geometric shape opening 202, thereby retaining the ability indicate the “outer” travel limit based on alignment of the outer peripheries of the stop element 206 and the top surface of the template 201. Thus, the user can confidently create a defect region cavity by utilizing the visual references, e.g., the travel indication feature, of the template 201 and cutter 204 to determine where a cut is being made relative to the template geometry.

With reference to FIG. 3, an exemplary embodiment of a template assembly 200 is depicted according to the present disclosure, generally including exemplary template 201, a mounting track 210, a locking screw 212, and a plurality of K-wires 213. Template 201 includes a peripheral template track 208, e.g., a recessed portion between the top and bottom surfaces of the template 201 for partially receiving placement of the mounting track 210 and anchoring the template 201 relative to an anatomical structure, e.g., a knee, and a plurality of pre-drilled holes 209 configured to receive and/or mate with the locking screw 212 (or a plurality of locking screws; not pictured). The peripheral template track 208 thereby provides flexibility to a user for anchoring the template 201 relative to an anatomical substrate by permitting about 360° of mounting access and ensures that the components of the template assembly 200 are properly oriented or aligned relative to a desired cavity region.

The mounting track 210 can be manufactured from a flexible yet durable material, e.g., rubber, and can be detachably secured relative to the template 201 by inserting a portion of the mounting track 210 into the peripheral template track 208 of the template 201 and inserting the locking screw 212, e.g., a set screw, thumb screw, and the like, into an appropriate pre-drilled hole 209, thereby detachably securing the mounting track 210 between the template 201 and the locking screw 212. Interaction between the locking screw 212 and a plurality of circumferentially spaced holes permits the mounting track 210 to be detachably secured to the template 201 at a variety of orientations and can be secured to a pre-drilled hole 209 by hand, thereby reducing the number of tools required for surgery. The mounting track 210 further includes a plurality of rows/columns of K-wire holes 211 for insertion of K-wires 213 in order to secure the template 201 relative to the cartilage 103 during use. In particular, the plurality of rows/columns of K-wire holes 211 can be oriented at varying angles relative to a mounting surface, thereby permitting the plurality of K-wires 213 to be inserted at varying angles for a more secure attachment to a desired anatomical location, e.g., to prevent motion of the template 201 during use.

Turning now to FIG. 4, the exemplary template assembly 200 is illustrated, including an exemplary cutter 204 approaching the template 201. The template assembly 200 of FIG. 4 is substantially similar to the template assembly 200 described with respect to FIG. 3 above. The cutter 204 is substantially similar to the cutter 204 described with respect to FIG. 2, generally including a drill bit 205 and a stop element 206, e.g., a bushing. As discussed above, the drill bit 205 can be, e.g., a downcutting drill bit as described in a U.S. patent application entitled “Orthopedic Downcutting Instrument and Associated Systems and Methods,” which published as US 2011/0238070 A1. FIG. 4 depicts the cutter 204 being lowered in the direction of the preformed geometric shape opening 202 in preparation for forming a defect region cavity.

With reference to FIG. 5, the drill bit 205 of the cutter 204 has been lowered/inserted into the preformed geometric shape opening 202 of the template 201 to form the defect region cavity in the cartilage 103. As discussed previously and as illustrated in FIG. 5, the alignment of a side surface of the stop element 206 of the cutter 204 and a template side surface 207 can indicate to a user that the drill bit 205 has reached the edge, i.e., an inner side surface, of the preformed geometric shape opening 202 of the template 201. This visual reference can be utilized by a user for awareness of where a cut has been made in the cartilage 103 relative to the opening formed in the template.

Turning to FIG. 6, an exemplary embodiment of a graft harvesting device 300 is provided, generally configured as a reusable portion A and a disposable portion B for harvesting a graft plug to fill the defect region cavity 314 formed in the cartilage 103 and manufactured from medically acceptable materials, e.g., stainless steel. In particular, the disposable portion B can be detachably connected to the reusable portion A and can be replaced by a customized disposable portion B, e.g., alternatively configured and/or dimensioned disposable portion B depending on the size and/or shape of the defect region 104 being operated on. On the other hand, the reusable portion A can generally be configured and dimensioned in a standard size to be functional in alternatively sized operations. The ability to reuse the reusable portion A thereby lowers the costs and the number of required instruments associated with the disclosed procedure relative to those taught in the prior art. Of note, the present disclosure is not limited by or to the reusable/disposable modality described above. Rather, it may be advantageous to supply detachably coupling subassemblies A and B that are both reusable and/or both disposable without departing from the spirit or scope of the present disclosure.

Still with reference to FIG. 6, the reusable portion A generally includes an elongated shaft 301, a handle 302, a top cap 303, a hammer mechanism 304 and a broach flange 306. In particular, the elongated shaft 301 can extend the length of the reusable portion A, thereby connecting the top cap 303 and the handle 302 at opposite ends relative to each other. The handle 302 can be securely/fixedly attached to the elongated shaft 301 and can include surface features, e.g., ridges, and/or be manufactured from a material permitting a strong and/or comfortable grasp by a user, e.g., foam, rubber, and the like. However, it should be understood that the top surface of the handle 302 located adjacent to the hammer mechanism 304 can be manufactured from a more durable material to permit hammering thereon. Further, the handle 302 can include a broach flange path 305 for axial translation of the broach flange 306, which will be described in greater detail below.

The hammer mechanism 304, e.g., a slap hammer, can freely slide axially along the elongated shaft 301 between the top cap 303 and the handle 302 and can be utilized for hammering, e.g., forcibly driving and/or axially applying a force, to the graft harvesting device 300 in a downward direction by hammering against the top surface of the handle 302 and/or in an upward direction by hammering against the bottom surface of the top cap 303. Thus, the hammer mechanism 304 permits the surgeon to apply an axial force to advance and/or withdraw the components of the disposable portion B into and/or from the cartilage 103 without accessing an auxiliary force-delivering device. The broach flange 306 can have a scalloped surface, and generally interlocks with the broach 312 to permit the broach 312 to be axially translated a maximum distance equal to the length of the broach flange path 305 and can further be rotated in a direction indicated by broach flange arrows 307 to lock and/or unlock the axial movement of the broach 312. The functionality of the broach flange 306 will be discussed in greater detail below with respect to the disposable portion B.

The disposable portion B of the exemplary graft harvesting device 300 generally includes a connecting shaft 309, a lower flange 308, a locking mechanism 310, a cutting member 311, a broach 312 and a plurality of elongated rod members 313. The configuration and/or dimensions of the components of the disposable portion B can be customized to meet the needs of a user based upon, e.g., the size and/or geometry of the defect region 104 of the cartilage 103 and/or the template 201 utilized. The connecting shaft 309 can be configured and dimensioned to interlock the reusable portion A components with the disposable portion B components in a mechanically functioning manner. Thus, the modular and/or disposable design of the disposable portion B permits the disposable portion B components to engage the reusable portion A, which can further be implemented for axially advancing and/or retracting the cutting member 311 and the broach 312 and for ejecting a graft plug post harvesting. In particular, the connection between the reusable portion A and the disposable portion B permits the broach 312 to mechanically interlock and/or interact with the broach flange 316 and further permits the cutting member 311 to rigidly interlock and/or interact with the handle 302. The modularity of the disclosed graft harvesting device 300 can reduce the costs associated with the replacement of instruments required for the procedures discussed herein relative to the procedures taught by the prior art.

Still with reference to the disposable portion B of FIG. 6, the cutting member 311 can be rigidly secured to the connecting shaft 309, can be defined by a hollow body and a serrated edge geometrically configured to match the preformed geometric shape opening 202 of the template 201, and can be used for harvesting a graft plug from a harvest location. In particular, the connecting shaft 309 can be defined by two concentric shafts, e.g., an inner and outer connecting shaft. The outer connecting shaft can rigidly secure the cutting member 311 to the elongated shaft 301 and/or the handle 302. The inner connecting shaft can be positioned inside the outer connecting shaft and can connect the broach 312 to the broach flange 306. Thus, the inner connecting shaft can be axially translated independently of and relative to the outer connecting shaft. Further, the broach 312 can be positioned within the hollow body of the cutting member 311, can be axially translated relative to the cutting member 311 and interlocks with the broach flange 306 for securely locking the broach 312 in a desired position. In particular, the broach 312 can be defined by, e.g., a rough surface, a plurality of downwardly and/or upwardly facing serrated edges/ridges, and the like, and can function as a reamer, thus permitting the user to “clean”, e.g., file away, provide finishing, and the like, the rough inner surfaces of the defect region cavity 314 potentially created by a cutter 204 to ensure a smooth fitting of the donor graft plug. The hammer mechanism 304 can be implemented to axially drive the broach 312 into the defect region cavity 314.

A plurality of elongated rod members 313 can be secured to the disposable portion B around the outer perimeter of the cutting member 311 and can function substantially similarly to the elongated rod members 101 of the trial member 100 as discussed with respect to FIG. 1. The elongated rod members 313 act as male components and can be inserted into the complementary female components located on the locking mechanism 310. Thus, as the graft harvesting device 300 is lowered against the cartilage 103 surface and the broach 312 is inserted into the defect region cavity 314, the elongated rod members 313 can be free to axially translate through the female components, e.g., apertures, of the locking mechanism 310 to capture the peripheral surface topography of the defect region cavity 314. However, the locking mechanism 310 acts substantially similarly to the locking mechanism 102 for securing the elongated rod members 313 in a position representative of the peripheral surface topography of the defect region cavity 314 in order to locate a topographically matching harvest location. The groove/track between the top and bottom portions of the locking mechanism 310 can further receive, e.g., a rubber band and/or ring element, for further frictionally locking the elongated rod members 313 in position. Thus, when an axial force is applied to the distal end of the elongated rod members 313, the elongated rod members 313 can translate axially through the complementary female components of the locking mechanism 310. However, when no axial force is applied to the elongated rod members 313, the locking mechanism 310 can secure the elongated rod members 313 in the most recent position. In addition, each of the plurality of elongated rod members 313 can further include an elongated rod member cap 315 to prevent the elongated rod members 313 from axially passing through and out of the locking mechanism 310. The lower flange 308 of the disposable portion B can be secured around and be axially translatable along the connecting shaft 309. In particular, the lower flange 308 can act as a “stop”, e.g., an even surface which provides a limit to the axial translation of the plurality of elongated rod members 313 in an upward direction. In addition, the lower flange can be translated down along the connecting shaft 309 to “reset”, e.g., reposition, the plurality of elongated rod members 313. Thus, by translating the lower flange 308 in a downward direction along the connecting shaft 309, a downward axial force can be applied to the elongated rod member caps 315 and thereby reposition the plurality of elongated rod members 313 in a desired position, e.g., a position of maximum extension below the locking mechanism 310.

Turning now to FIG. 7, the exemplary graft harvesting device 300 is depicted in preparation for insertion of the broach 312 into the defect region cavity 314. In particular, the broach flange 306 has been translated along the broach flange path 305 to the lowest portion of said path, thereby axially translating the broach 312 from a position inside the body of the cutting member 311 to a position protruding a predetermined distance out of the body of the cutting member 311, and has been rotated in the appropriate direction to lock the broach 212 in the protruding position. The predetermined distance which the broach 312 protrudes out of the cutting member 311 generally corresponds to the depth of the donor graft plug required to fill the defect region cavity 314. The plurality of elongated rod members 311 are axially translated to a position of maximum extension below the locking mechanism 310, e.g., the elongated rod member caps 315 abut the top surface of the locking mechanism 310 and the elongated rod members 311 extend below a bottom surface of the broach 312. Thus, as the broach 312 is lowered into the defect region cavity 314, the elongated rod members 311 contact the cartilage 103 surface periphery surrounding the defect region cavity 314 prior to the broach 312 contacting the defect region cavity 314. This ensures that an accurate peripheral surface topography is obtained by the elongated rod members 311 simultaneously to the “cleaning” of the surfaces of the defect cavity region 314. The broach 312 can be axially driven into the defect region cavity 314 by hammering the hammer mechanism 304 against the top surface of the handle 302.

With reference to FIG. 8, the broach 312 of the exemplary graft harvesting device 300 has been inserted into the defect region cavity 314 and the plurality of elongated rod members 313 have simultaneously captured the peripheral surface topography of the defect region cavity 314. In particular, as the graft harvesting device 300 is lowered and/or hammered into the defect region cavity 314, except for the elongated rod members 313, all of the components of the disposable portion B remain axially fixed relative to each other.

Turning to FIG. 9, the broach 312 of the exemplary graft harvesting device 300 has been removed from the defect cavity region 314. In general, the removal of the broach 312 from the defect cavity region 314 can be performed by, e.g., pulling on the handle 302 in an upward direction away from the defect cavity region, utilizing the hammer mechanism 304 to hammer against the top cap 303, and the like. Further, as the broach 312 is removed from the defect region cavity 314, all of the components of the disposable portion B remain axially fixed relative to each other. Thus, as can be seen in FIG. 9, after removal of the broach 312 from the defect region cavity 314, the plurality of elongated rod members 313 remain fixed by the locking mechanism 310 in a position representative of the peripheral surface topography of the defect cavity region 314.

With reference to FIG. 10, once the defect region cavity 314 has been “cleaned” and the peripheral surface topography of the defect cavity region 314 has been obtained, the broach 312 can be retracted axially in an upward direction by unlocking and translating the broach flange 306 to the highest position along the broach flange path 305. In particular, the broach flange 306 can be spring-loaded to automatically axially translate the broach flange 306 to the highest position along the broach flange path 305 when the broach flange 306 has been unlocked from a protruding position. However, it should be understood that the exemplary broach flange 306 can also be manually translated to the highest position along the broach flange path 305 and the broach 312 can be locked in a retracted position by, e.g., rotating the broach flange 306 along a broach flange locking path (not shown) similar to the broach flange locking path 316 located at the lowest position along the broach flange path 305. The broach flange locking path 316 can be defined by a path oriented at about a 90° angle relative to the broach flange path 305 along which the broach flange 306 can rotate, thus preventing the broach flange 306 from entering and being translatable along the broach flange path 305.

The retraction of the broach 312 enables the user to implement the peripheral surface topography of the defect region cavity 314 captured by the plurality of elongated rod members 313 to identify and/or locate, e.g., match, a harvest location having a complementary surface topography. This procedure can be implemented in conjunction with the trial member 100 previously discussed with respect to FIG. 1. Once a complementary surface topography of a harvest location has been located, the user can utilize the hammer mechanism 304 to axially drive the cutting member 311 downward into an allograft and/or autograft donor location for harvesting a donor graft plug. It should be understood that the donor location may be, e.g., autograft, allograft, xenograft, synthetic, and the like. The length of the cutting member 311 can be a predetermined and customized length based on the depth of the defect region cavity 314. In addition, the retracted distance of the broach 312 into the cutting member 311 ensures that the depth of the inner cavity of the cutting member 311 is complementary to the depth of the defect region cavity 314. Thus, as the user axially drives the cutting member 311 into the allograft and/or autograft donor location, when the bottom surface of the broach 312 contacts the top surface of the allograft and/or autograft donor location, the cutting member 311 is prevented from moving further into the allograft and/or autograft donor location and the user can understand that the desired predetermined height of the harvest graft plug has been reached. Once the desired harvest graft plug height has been reached, the cutting member 311 can be removed from the allograft and/or autograft donor location with the harvest graft plug located inside the cavity of the cutting member 311 by utilizing the hammer mechanism 304 to axially hammer against the top cap 303.

Turning now to FIG. 11, the exemplary graft harvesting device 300 is illustrated after removal from the allograft and/or autograft donor location, including a bottom portion of the donor graft plug 317 protruding out of the cutting member 311. As discussed above, the inner cavity of the cutting member 311 defines the geometrical shape and height required for the donor graft plug 317 to fill the defect region cavity 314. Thus, the portion of the donor graft plug 317 protruding past the lowest point of the cutting member 311 defines additional and/or unwanted graft material which can result during removal of the cutting member 311 from the donor location. In particular, the additional and/or unwanted graft material is to be removed prior to inserting the donor graft plug 317 into the defect region cavity 314, while the top surface of the donor graft plug 317 located in the cavity of the cutting member 311 is defined by the desired surface topography.

With reference to FIG. 12, in order to remove, e.g., trim, the additional and/or unwanted graft material from the donor graft plug 317 and thus create a desired donor graft plug 317 depth dimension, an exemplary cutter guide 318 can be implemented. In particular, the exemplary cutter guide 318 generally includes an attachment member 319, a connecting member 320, a cutter guide head 321 and a cutter guide channel 322. The attachment member 319 can be configured and dimensioned as, e.g., a flexible C-shaped clip with a spring-like property, thus permitting a user to fit and secure the attachment member 319 relative to and/or around the elongated shaft 301 and/or the handle 302. The spring-like property of the attachment member 319 can be sufficiently strong to prevent unwanted motion of the cutter guide 318 during trimming of the donor graft plug 317. Although illustrated as a detachable member, it should be understood that the cutter guide 318 can also be configured as an integral component of the reusable portion A.

The connecting member 320, e.g., the arm, can connect the attachment member and the cutter guide head 321 and can be configured and dimensioned to extend the cutter guide head 321 over the components of the disposable portion B and align the cutter guide channel 322 with the distal end of the cutting member 311. It should be understood that the connecting member 320 can be configured as a telescoping connecting member 320, thus permitting a user to vary the length of the connecting member 320 as needed depending on the configurations and/or dimensions of the disposable portion B components. Further, the cutter guide head 321 includes a cutter guide channel 322 for passing through and aligning a trimming instrument 323, e.g., a saw, with the distal end of the cutting member 311, thereby permitting an accurate trimming of the unwanted graft material of the donor graft plug 317 and ensuring a desired donor graft plug 317 depth of, e.g., about 10 mm, depending on the defect region 104 and/or joint being repaired. It should be understood that alternative desired depths can be obtained by implementing appropriately customized components of the disposable portion B, e.g., a depth of about 6 mm for a shoulder joint. Due to the desired donor graft plug 317 depth being located inside the cavity of the cutting member 311, other than aligning the trimming instrument 323 with the distal end of the cutting member 311, the user is generally not required to axially size the depth of the donor graft plug 317. Further, the implementation of the cutter guide 318 in conjunction with the desired donor graft plug 317 depth being located inside the cavity of the cutting member 311 eliminates the need for utilizing, e.g., a chisel, which could potentially dislodge the donor graft plug 317 from the graft harvesting device 300 and result in an inaccurate donor graft plug 317 geometry.

With reference to FIG. 13, the ejection of the donor graft plug 317 from the cutting member 311 of the exemplary graft harvesting device 300 is illustrated. In particular, while the donor graft plug 317 is located in the inner cavity of the cutting member 311, the broach flange 306 can be unlocked and translated down the broach flange path 305, thereby axially translating the broach 312 through and out of the inner cavity of the cutting member 311. The pressure from the translating broach 312 acts to eject the donor graft plug 317 out of the cutting member 311. It should be understood that the broach 312 can be implemented to eject the donor graft plug 317 out of the cutting member 311 for independent insertion into the defect region cavity 314 and/or the donor graft plug 317 can be ejected out of the cutting member 311 directly into the recipient site, e.g., the defect region cavity 314. Implementing the large surface area of the bottom of the translating broach 312, rather than an additional instrument, e.g., a small rod with a single point of force application, provides a substantially even force distribution on the top surface of the donor graft plug 317, thus preventing damage to the donor graft plug 317 during ejection from the graft harvesting device 300. In particular, the improved force distribution from the broach 312 onto the top surface of the donor graft plug 317 prevents damage to the desired surface topography of the donor graft plug 317 during ejection.

Turning now to FIG. 14, an alternative exemplary embodiment of a reusable portion A′ of a graft harvesting device 300′ is presented. In particular, the exemplary graft harvesting device 300′ generally includes the reusable portion A′ of FIG. 14 and the disposable portions B′ of FIGS. 15A and 15B. The reusable portion A′ of FIG. 14 is configured and functions substantially similarly to the reusable portion A discussed previously, generally including an elongated shaft 301′, a handle 302′, a top cap 303′ and a hammer mechanism 304′. The reusable portion A′ can further include an integral cutter guide 318′ configured as an attachment member 319′, e.g., a hinge, a connecting member 320′, a cutter guide head 321′ and cutter guide channel 322′. It should be understood that the connecting member 320′ can be configured as a telescoping connecting member 320′, thus permitting a user to vary the length of the connecting member 320′ as needed depending on the configurations and/or dimensions of the disposable portion B′ components. The spring-loaded button 306′ can function substantially similarly to the broach flange 306 of FIG. 6. In particular, the spring-loaded button 306′ can be actuated to, e.g., retract and/or protrude the broach 312′, eject the donor graft plug 317, reset the plurality of elongated rod members 313′ with a lower flange (not shown) similar to the lower flange 308 discussed above, and the like. The reusable portion A′ components can be detachably secured, e.g., mechanically interlocked, to the disposable portion B′ components through a mechanical connection, including pins 324′ and a shaft aperture 325′.

With reference to FIGS. 15A and 15B, an alternative exemplary embodiment of a disposable portion B′ is presented. In particular, the assembly of the cutting member 311′, the plurality of elongated rod members 313′, the locking mechanism 310′ and the broach 312′ generally represent the disposable portion B′ according to the present disclosure. The cutting member 311′, plurality of elongated rod members 313′ and the locking mechanism 310′ of FIG. 15A are substantially similar to those discussed previously. It should be noted that FIG. 15B further illustrates the addition of a locking element 326′, e.g., a rubber band and/or O-ring, fitted into the groove between the top and bottom surfaces of the locking mechanism 310′. As previously mentioned, the locking element 326′ frictionally prevents the axial motion of the plurality of elongated rod members 313′. The broach 312′ of FIG. 15B is substantially similar to the broach 312 discussed previously, further including a broach shaft 327′ for mating, e.g., mechanically interlocking, with the shaft aperture 325′ of the reusable portion A′.

In accordance with yet another embodiment of the present disclosure, a method for defect repair is provided, generally including the steps of establishing a referential orientation of an instrument relative to an anatomical location, capturing an entire surface topography of the anatomical location of the defect region, forming a defect region cavity of a predefined geometry in the anatomical location, and using the captured entire surface topography of the anatomical location of the defect region to identify a donor location with a complementary surface topography as a harvest region for a plug to fill the defect region cavity. The defect region cavity is generally formed with a predefined depth and is formed at substantially a right angle relative to the axis of the instrument used to form such defect region cavity. The exemplary method generally further includes using a detachable broach member for cleaning the defect region cavity and using a plurality of elongated rod members for capturing a peripheral surface topography of the anatomical location in proximity to the defect region cavity. Further still, the exemplary method generally includes obtaining a plug from the harvest region, using a cutter guide to trim the plug to a predefined depth, using a detachable broach member to eject the plug from a cutter, and introducing the plug into the defect region cavity. In general, the defect region cavity is formed using a template having a predefined opening geometry, the plug is obtained using the cutter having a cutting geometry, and the predefined opening geometry of the template and the cutting geometry of the cutter correspond to each other.

Turning now to FIG. 16, a flowchart is provided of an exemplary method for defect repair implementing the exemplary trial members and graft harvesting members discussed herein. In particular, the exemplary method includes establishing a referential orientation of an instrument to be implemented in conjunction with the exemplary trial members and graft harvesting members relative to an anatomical location (400). The trial member 100 can further be utilized to capture an entire surface topography of the anatomical location of a defect region 104 (401). A defect region cavity 314 can be formed with a predefined geometry in the anatomical location (402). A donor harvesting location can be identified as an appropriate harvest region for a donor graft plug 317 to fill the defect region cavity 314 based on a complementary surface topography (403). The defect region cavity 314 can be cleaned and simultaneously the peripheral surface topography of the anatomical location in proximity, e.g., surrounding, the defect region cavity 314 can be obtained (404). A donor graft plug 317 with a complementary entire and peripheral surface topography can be obtained from a harvest region (405). The donor graft plug 317 can be trimmed to a predetermined depth/height to property fill the defect region cavity 314 (406). The donor graft plug 317 can be ejected out of the cutting member 311 of a graft harvesting device 300 (407). The donor graft plug 317 can then be introduced into the defect region cavity 314 (408).

The exemplary instruments, methods and systems may be used in connection with mapping techniques and systems discussed in PCT applications entitled “Systems, Devices and Methods for Cartilage and Bone Grafting” and “Instruments, Methods and Systems for Harvesting and Implanting Cartilage Materials,” which published as WO 2009/154691 A9 (corrected version) and WO 2011/008968 A1, respectively. Thus, in exemplary embodiments of the present disclosure, a clinician may be guided in his use of the disclosed instruments and systems by cartilage surface mapping data in locating/identifying harvest sites for “best fit” grafts, i.e., grafts that exhibit desired geometric and/or surface attributes for use in particular implantation site(s). Alternatively, the disclosed instruments, methods and systems may be employed to access anatomical sites independent of such mapping techniques/systems.

With reference to FIG. 17, an exemplary schematic diagram is shown of a surface mapping system 500 to acquire data regarding cartilage and/or bone anatomies and to enable identification of suitable donor sites to harvest cartilage to repair defects in a patient's bone. The exemplary system 500 generally includes an imaging apparatus 501 to capture image data of an area on a patient's body, e.g., a patient's foot 502, which includes at least one of the defect regions and an area around the defect. The imaging apparatus 501 includes, e.g., one or more of a Magnetic Resonance Imaging (MRI) system, a computed tomography apparatus configured to generate three-dimensional images from a series of two-dimensional images (e.g., X-Ray) taken around a single axis of rotation, a Medical sonography (ultrasound) imaging device, and any other suitable imaging device to acquire data representative of anatomical structures in a patient's body. In some exemplary embodiments, the data relating to the defect region of the patient may have been acquired at an earlier time and/or at some location other than where the system 500 is located, in which case, the data may be received at the system 500 from some remote location which can electronically communicate, e.g., via one or more types of communication networks such as a network 503, including the Internet, a telephony network, and the like, the data relating to the defect region of the patient.

The imaging apparatus 501 acquires one or more images of a site in the body of a patient who has the defect, e.g., the talus surface at the foot 502, requiring a cartilage-bone graft procedure to correct. In some exemplary embodiments, the mapping system 500 also includes an optional signal processing unit 504 connected to the imaging apparatus 501. The processing unit 504 receives the signals communicated from the imaging apparatus 501 and performs signal processing and/or enhancement operations. Signal enhancement operations may include, e.g., amplification, filtering, and the like. For example, the processing unit 504 may be configured to perform noise reduction to remove noisy artifacts from acquired image data. Other types of processing may include image processing operations to transform the image data into resultant data which can be more easily manipulated for the purpose of identifying donor sites. For example, the acquired data can be processed to generate a surface model corresponding to the defect region and/or the area proximate the defect region, transform spatial representations into another domain, e.g., the frequency domain, which is more conducive for various types of processing, and the like.

The processed data can subsequently be communicated to the controller processor 505. The controller processor 505 includes a storage device 506 to store the data (processed and/or raw acquired data) relating to the defect region of the patient, and to store a donor database 507 which includes information on each of a plurality of donor sites of the body. As will become apparent below, the database 507 may be constructed based on data acquired from multiple sources and/or multiple specimens. The acquired data may be used to develop and/or expand the database 507 and enhance the sensitivity and specificity of the system 500. Typically, the data stored on the database 507 pertains to healthy, non-injured specimens (or a composite representation thereof), thus enabling identification of suitable healthy sites in the body from which bone and/or cartilage can be harvested to perform bone-cartilage grafts. The controller processor 505 can thus be configured to receive a first data relating to a defect region of a patient and to identify, based on the received first data, at least one donor site from the donor database 507 from which it can harvest a graft of bone and cartilage to repair the defect region of the patient.

In some exemplary embodiments, the data stored on the database 507 includes information related to healthy, non-injured specimens of like and unlike joint donor sites relative to the joint having a defect region to be repaired. For example, if the defect region is located at a joint in the toe of the patient, the database 507 can include information related to healthy donor sites from which bone and/or cartilage can be harvested located in toe joints, i.e., like joints, and can further include information related to healthy donor sites from which bone and/or cartilage can be harvested located in joints in other parts of the body, e.g., the knee, talus, shoulder, clavicle, and the like (i.e., unlike joints). To determine the compatibility of an unlike joint for the defect region, the database 507 may include correlation information indicating which like joints of the body can be used for harvesting of bone-cartilage grafts to be implanted in different unlike joints of the body based on anatomical characteristics of the joints, e.g., cartilage thickness, cartilage density, cartilage resiliency/durability, and the like. The correlation information can be based on, e.g., prior determinations of the anatomical characteristics of joints, support in the literature known in the industry, expert validations of the unlike joints, combinations thereof, and the like. The information stored in the database 507 for both like and unlike joints can include imaging and non-imaging data, e.g., imaging data of the donor site, imaging data of an area surrounding the donor site, sizing of the donor site, the surface topography of the donor site, anatomical characteristics of the donor sites, and the like. The data acquired by the processing device 505 for the defect region can be substantially similar imaging and/or non-imaging data such that the processing device 505 can determine the compatible donor sites for the defect region.

As a further example, if the defect region is located in joint “A” of the patient, the correlation information stored in the database 507 can be utilized by the processing device 505 to determine that unlike joints “B” and “C” are available as compatible donor sites for harvesting a bone-cartilage graft to repair the defect region. In some exemplary embodiments, the processing device 505 can perform a prioritization of the unlike joints compatible with the defect region to determine which unlike joint should be used for the bone-cartilage graft. For example, joint “A” may be located in the toe of a patient and the unlike joints “B” and “C” found to be compatible with joint “A” may be located in the knee and talus, respectively. The correlation information may further indicate that while joint “B” is compatible with joints of the toe, knee, shoulder and clavicle, joint “C” is only compatible with joints of the toe and talus. To ensure efficiency in the selection and/or availability of unlike joints, the processing device 505 can utilize the correlation information to determine that due to the narrow compatibility of joint “C” with other joints, joint “C” should be used for harvesting of a bone-cartilage graft to repair the defect region of joint “A”. Since joint “B” has a broader compatibility with other joints, it can be stored in the database 507 and implemented for harvesting a bone-cartilage graft at a subsequent time.

In some embodiments, the storage device 506 hosting the donor database 507, or another storage device hosting the database 507, may be located at one or more remote locations which may be accessed by multiple systems, such as the mapping system 500. Thus, such a remote device may serve as a central data repository on which data pertaining to donor sites may be stored. A user locally interacting with the system 500 may therefore access remotely, via a network 503, a database such as the database 507 to retrieve data as required. For example, and as will be described in greater details below, data pertaining to potential donor sites which is compared to data relating to a defect region can be retrieved from a remote location. Optionally, a 3D printer 508 may be locally or remotely interconnected to the controller processor 505. Such a 3D printer 508 may be used to create 3D custom templates corresponding to any identified donor site and/or to the defect region.

In some implementations, the controller processor 505 may also be configured to perform learning functions. A machine learning system is generally a system which iteratively analyzes training input data and the input data's corresponding output, and derives functions or models that cause subsequent inputs to produce outputs consistent with the machine's learned behavior. Thus, in some embodiments, the controller processor 505 may be configured to perform learning functions which include, e.g., identifying the type of donor site corresponding to newly received data, classifying the data so it is associated with other data sets corresponding to the same anatomical locations, automatically selecting several potentially suitable donor sites for further processing with respect to data received regarding the defect region, and the like. Some implementations of learning functionalities may be performed using, e.g., a neural network system implementation. A neural network includes interconnected processing elements (effectively the system's neurons), whose connections can be varied, thus enabling the neural network to adapt (or learn) in response to training data it receives. In some embodiments, a learning system may be implemented using decision trees, e.g., a graph of decisions/actions and their possible outcomes. A decision tree takes as input an object or situation described by a set of properties, and outputs a decision, i.e., an outcome. Alternatively and/or additionally, in some embodiments, the learning system may be implemented using regression techniques. Regression techniques produce functions, e.g., curves, which best fit a given set of data points. These curves can subsequently be applied to input data to determine the output based on the derived curves. Derivation of best fit curves is typically the solution to optimization problems, in which a particular error measure, e.g., least-square error, is being minimized. Other types of learning system implementations may also be used.

With reference to FIG. 18, a schematic diagram of a generic computing system 550 is provided which may be used to implement the controller processor 505 and/or the signal processing unit 504. The computing system 550 includes a processor-based device 551, e.g., a personal computer, a specialized computing device, and the like, which typically includes a central processor unit (CPU) 552. In addition to the CPU 362, the system 550 includes main memory, cache memory and bus interface circuits (not shown). The processor-based device 551 includes a mass storage element 553, which may be the same device or a separate device from storage device 506. The mass storage element 553 may be, e.g., a hard drive associated with personal computer systems. The computing system 550 may further include a keyboard 554, a monitor 555, e.g., a cathode ray tube (CRT) or liquid crystal display (LCD) monitor.

The processor-based device 551 can be configured to facilitate, e.g., the implementation of the data capture and/or mapping operation used to identify suitable donor sites for harvesting a graft of bone-cartilage as described herein. The storage device 553 may thus include a computer program product which, when executed on the processor-based device 551, performs operations to facilitate the implementation of the data capture, mapping and/or site identification procedures described herein. The processor-based device 551 may further include peripheral devices to enable input/output functionality, e.g., a CD-ROM drive, a flash drive, a network connection, and the like, for downloading related content to the connected system. Such peripheral devices may also be used for downloading software containing computer instructions to enable general operation of the respective system/device, as well as data from remote locations, e.g., donor site data. Alternatively and/or additionally, in some exemplary embodiments, special purpose logic circuitry, e.g., a field programmable gate array (FPGA) or an application-specific integrated circuit (ASIC) may be used in the implementation of the system 550. Other modules which may be included with the processor-based device 551 are speakers, a sound card, a pointing device, e.g., a mouse, a trackball or a touch-based graphical user interface (GUI), by which the user can provide input to the computing system 550, and the like. The processor-based device 551 may include an operating system, e.g., Windows XP® Microsoft Corporation operating system. Alternatively, other operating systems could be used. Additionally or alternatively, one or more of the procedures performed by the signal processor 504 and/or the controller processor 505 may be implemented using processing hardware, such as digital signal processors (DSP), field programmable gate arrays (FPGA), mixed-signal integrated circuits, and the like.

The various systems and devices constituting the system 500 may be connected using conventional network arrangements. For example, the various systems and devices of system 500 may constitute part of a public private packet-based network, e.g., the Internet. Other types of network communication protocols may also be used to communicate between the various systems and devices. Alternatively, the systems and devices may each be connected to network gateways which enable communication via a public network, such as the Internet. Network communication links between the systems and devices of system 500 may be implemented using wireless or wire-based links. For example, in some embodiments, the controller processor 505 may include a communication apparatus, e.g., an antenna, a satellite transmitter, a transceiver such as a network gateway portal connected to a network, and the like, to transmit and/or receive data signals. Further, dedicated physical communication links, such as communication trunks, may be used. Some of the various systems described herein may be housed on a single processor-based device, e.g., a server, configured to simultaneously execute several applications.

Referring to FIG. 19, a flowchart of a procedure 600 to identify suitable donor sites for bone-cartilage grafts for repairing a defect region of a patient is shown. Initially, a computing device, such as a computer or the controller processor 505 depicted in FIG. 17 accesses a donor database 507 which includes information on each of a plurality of donor sites of like and/or unlike joints that may have been compiled and/or evolved from several sources of data (601). In some exemplary embodiments, the database 507 may have been populated with data downloaded, or otherwise retrieved, from remote locations which maintain data regarding potential donor sites. In some exemplary embodiments, the data may have been obtained by acquiring raw data, e.g., image data obtained using conventional imaging techniques, such as MRI imaging, CT imaging, ultrasound imaging, laser scans, and the like, from specimens having healthy cartilage of specific anatomical locations, e.g., joints which do not have defects or are otherwise non-injured. For example, in some embodiments, data acquired using a large sample of individuals may be used to assemble data about possible donor sites in those individuals, including data representative of the topology, health and other physiological attributes, e.g., gender, age, race, activity index, BM1, V02, cartilage thickness, bone density, and the like, of those donor sites. Such data acquired using such a sample of individuals may include data about some or all feasible sites in a body from which bone and/or cartilage may be harvested. Accordingly, the individuals used to acquire this data may be put through a comprehensive and systematic protocol of data acquisition procedure such that data regarding all (or substantially all) possible donor sites is acquired. For example, the data acquisition stage required for constructing the database may require that all the joint areas in a person's feet, knees and/or shoulders be imaged using one or more imaging devices and/or surveyed using non-imaging type devices, e.g., devices utilized to measure bone density, to obtain an accurate and comprehensive database 507. Data processed in this manner can be added to the donor database 507. As noted, in some embodiments, a learning system, e.g., implemented on the controller processor 505 or on some other dedicated processing device, may be used to process acquired data of graft sites (e.g., donor sites and/or recipient sites) which is to be added to the database 507. For example, such a learning system may be used to determine (through implemented classification functions) the identity of the site with respect to which data was received, facilitate the identification procedure to identify donor sites which would be suitable for harvesting bone-cartilage to repair the particular damaged site, and the like.

The donor sites with respect to which data is acquired and added to the database 507 include donor sites of like and/or unlike joints and different shapes and sizes, including donor sites suitable for harvesting non-cylindrical bone-cartilage grafts. The data for those donor sites can subsequently be used to identify suitable donor sites from which non-cylindrical bone-cartilage grafts can be harvested. Particularly, the systems described herein enable matching irregularly shaped defects of the damaged/injured recipient site(s) to available donor sites which can be used to harvest non-cylindrical bone-cartilage grafts. Conventional bone-cartilage grafting systems and methods typically extract grafts having standard shapes, e.g., cylindrical, thus limiting the repertoire of available donor sites, e.g., donor sites from which such standard shaped grafts can be harvested. Once suitable donor sites are identified, whether from like joints or unlike joints, various types of grafts can be harvested, including standard-shaped grafts, e.g., cylindrical grafts, as well as irregularly-shaped grafts. Harvesting irregularly shaped grafts can be performed using a set of predetermined irregularly shaped templates or, in some exemplary embodiments, by generating custom templates.

In some exemplary embodiments, the specimens used to acquire data to populate the donor site database 507 may include cadavers. Under those circumstances, more invasive data acquisition procedures may be used to acquire the data. For example, in some embodiments, one or more of a cadaver's joints may be disarticulated to expose the actual cartilage tissue. With the joint sites of the cadavers disarticulated, a high resolution image scanner may be used to scan the tissue to obtain an accurate representation of the cartilage tissue. A suitable laser scanner to scan exposed cartilage may be, e.g, a NextEngine 3D Scanner manufactured by NextEngine, Inc. Other laser scanners and/or other types of high quality image capture devices may be used.

In some exemplary embodiments, data acquired from multiple specimens, e.g., live individuals and/or cadavers, may be used to generate a composite representation of donor sites. For example, the data acquired may be averaged to obtain a general representative model of the plurality of donor sites. In some variations, several representative models of donor sites and their associated data may be generated from multiple specimens that each correspond to a particular individual type such that, when identification of a suitable donor site is undertaken, a model which is more representative of the particular traits of the patient for whom a bone-cartilage graft is required can be used. For example, different general model sets of donor sites may be constructed for male and female models.

In circumstances where the database 507 is constructed, at least partly, by collecting data about donor sites (and areas surrounding such donor sites) from specimens, a system arrangement similar to the arrangement depicted in FIG. 17 may be used. Thus, such an arrangement would include an imaging apparatus 501, e.g., a NextEngine laser scanner, to capture data. The captured data would be forwarded to a signal processing unit 504, which may be implemented as a processor-based computing device to perform digital processing, e.g., filtering, on the data and/or a dedicated processing device to perform some or all of the processing operations. A storage device 506 to store captured and/or processed data may also be provided. In some embodiments, such storage device 506 can be locally connected to a processor-based computing device which may also serve to perform data processing, perform database management operations, e.g., by executing database management tools, and to perform the donor site identification procedure to identify suitable donor sites from which bone-cartilage may be harvested.

With reference to FIG. 20, an exemplary arrangement of a system 700 to acquire, process and/or store data is shown. The system 700 includes a laser scanner 701, e.g., a NextEngine scanner, whose imaging port, i.e., an outlet through which laser radiation is directed at the object being scanned, is facing an object 702, e.g., a disarticulated body joint. Data acquired by the imaging apparatus 701 can be communicated to a processing apparatus 703, e.g., a computer. The processing apparatus 703 typically includes software implemented applications to interface and/or interact with imaging apparatus 701 and may perform preliminary processing on data communicated by the imaging apparatus 701, e.g., perform analog-to-digital conversion, down-sample the data, and the like. The processing apparatus 703 may also run software-based implementations of data processing applications, e.g., SolidWorks 3D CAD software applications, and the like. Further, the processing apparatus 703 may also include software implementations to perform the donor site identification procedure described herein. Data captured and processed may be maintained in a database implemented on the processing apparatus 703 or may be stored on a remote storage device and processing center implemented, for example, on a remote server connected to the processing apparatus 703 via a communications network.

Data acquired by imaging apparatus 701 for populating the donor site database may be processed to, for example, remove noisy artifacts from the image, remove unnecessary data, perform various mathematical mapping and/or transformation operations (e.g., normalization operations, re-sizing/scaling operations so all data corresponds to features at the same scale, frequency domain transformations, and the like) to transform the data into formats which are more conducive for subsequent search operation on the database. As noted, further processing on the image data (including image data on which some preliminary processing such as noise filtering and/or artifact removals have already been performed) can be performed on the data to convert it into a format which can subsequently be more easily controlled and can be more conducive for performing the donor site identification procedure described herein, e.g., using a format which enables comparisons of different donor site surfaces to one another. In some implementations, the data acquired can be used to generate surface models representative of the donor sites. The surface model may include data regarding the topology of the area, as well as other information descriptive of the area, e.g., bone thickness, bone density, and the like.

Several procedures may be used to generate the surface models. For example, in some exemplary embodiments, the captured data of the defect region can be provided as input to various computer aided design (CAD) interface applications, e.g., the SolidWorks 3D CAD application developed by Dassault Systemes SolidWorks Corp., and the like, such that the application generates a 3D rendering corresponding to the data provided. Specifically, the point cloud of data representative of an acquired image can be incorporated into SolidWorks (or any other CAD application used) to generate a resultant surface model. This data can then be stored in a format compatible with the graphical representation rendering or may be converted and stored using another type of representation of the surface model features, e.g., a representation of a composite of graphical primitives corresponding to, for example, dimensions and curvatures of lines or segments of the surface model, and the like. The generated surface model may be compared with, for example, a surface model representative of the damaged cartilage/bone of a defect region, to determine if the potential donor site would be suitable for harvesting bone-cartilage to repair the defect region of the patient. Surface comparisons may be performed visually by the operator of the system, e.g., a surgeon, who examines the surfaces compared to each other and selects one position/orientation which appears to result in the best match, or via a processing device. The procedure of matching the model surface of the defect region to model surfaces of potential donor sites can be repeated for other donor/recipient sites.

In some exemplary embodiments, the generated surface model of the donor site may be further manipulated to fit the surface model into a corresponding bone structure to provide further details on the anatomical structure of the potential donor site and provide orientation context to the user on how the surface model is overlaid relative to the bone structure. In some embodiments, the model representation of the bone structure on which the cartilage surface model is overlaid may have been acquired from other specimens, i.e., not necessarily from the same individual whose cartilage data was acquired, using an imaging device, such as an MRI imaging apparatus, a CT imaging apparatus and/or a laser scanner. Under such circumstances, when a generated surface model of the cartilage is overlaid on a previously acquired or imported model of the bone structure, small anatomical differences between the two models may be evident, e.g., topographical differences, size differences, and the like. Alternatively and/or additionally, in some embodiments, the bone structure models and the cartilage models may have been derived from the same set of specimens.

With reference again to FIG. 19, the procedure 600 further includes receiving a first data set relating to a defect region of a patient (602). As noted, the defect region includes an area of a bone, a portion of which includes at least one of a missing and/or damaged cartilage, e.g., hyaline cartilage, and the like. In some embodiments, the data relating to the defect region may be representative of at least one of, for example, the defect region and the area around the defect region. Such data may be acquired by using imaging techniques, such as MRI, CT, ultrasound, and the like, as noted previously, to image the area and construct, using the data, a surface model. In some exemplary embodiments, the data relating to the defect region may be representative of anatomical characteristics of the defect region and/or healthy cartilage surrounding the defect region. Such data may be acquired by using non-imaging techniques. The data may have been sent via a communications link by a health professional, such as the patient's physician or the surgeon who will perform the graft. The surface model may include data regarding the topology of the area, as well as other information descriptive of the area, e.g., bone thickness, bone density, and the like. As noted with respect to the procedure for generating surface models for the potential donor sites, several procedures may be used to generate the surface model, including, e.g., using the Pro/Engineer CAD application developed by Parametric Technology Corporation, MA, the SolidWorks 3D CAD application developed by Dassault Systemes SolidWorks Corp., and the like, to generate a 3D rendering corresponding to the received first data corresponding to the defect region and the area surrounding it. As further noted, the data can then be stored in a format compatible for providing graphical representations of the rendering or may be converted and stored as numerical representations of the surface model features, e.g., be represented as primitives corresponding to dimensions and curvatures of lines or segments of the surface model. Based on the captured data, a surface model of the cartilage can be generated (and in some embodiments, a model for the bone structure can also be generated) in a manner similar to that used for the surface model and bone structure models populating the donor database. This surface model may subsequently be manipulated, e.g., rotated, sized, and the like, during the donor site identification procedure to compare the defect region to donor models in the donor database.

In some exemplary embodiments, the received data relating to the defect region of the patient can be used to identify data in the donor database corresponding to the patient's defect. In other words, instead of using the data relating to the defect region to identify a donor site by comparing the data of the defect region to the donor data in the database, the data relating to the defect region can be used to first identify a corresponding non-damaged cartilage structure, i.e., the counterpart healthy cartilage from the donor database which does not have a defect, which can subsequently be used to identify a suitable donor site at a like or unlike joint to harvest bone-cartilage to repair the defect region.

With continued reference to FIG. 19, once the first data has been received and/or the data was used to generate a surface model to be used in identifying a suitable donor site or to first identify a corresponding healthy cartilage-bone counterpart from the donor database, at least one donor site of a like or unlike joint from the donor database can be identified based on the first data relating to the defect region (603). Identifying the at least one donor site may include performing comparisons of the data representative of, for example, surface models of donor sites from the database to the first data relating to the defect region, anatomical characteristics of the donor sites from the database relative to the anatomical characteristics of the defect region, and/or to some derivative data thereof (for example, a generated surface model for the defect region, a surface model of the same anatomical location but without the defect, or a relevant portion of whichever surface model is selected for performing the comparisons, e.g., only the area in the surface model which includes the defect region). Based on these comparisons, the at least one suitable donor site can be determined.

Further, in some exemplary embodiments, the surface model data obtained from the data relating to the defect region can be used to compare, for example, the dimensions and surface curvatures of the model, to the corresponding dimensions and curvature data of the plurality of donor sites in the donor database. The two surface models can be similarly scaled and/or directionally tagged to enable an accurate comparison. The dimensions and curvatures can thus be compared to determine if the particular cartilage would be a suitable donor site to harvest bone-cartilage to repair the defect region in the body of the patient.

In performing the comparisons to identify suitable donor sites, the model surfaces can be manipulated to place them in different orientations to facilitate the comparisons. In particular, the surface model corresponding to the defect region can be rotated relative to the donor site surface models to determine an optimal matching orientation for the models being compared. For example, the surface model of the defect region can be rotated to determine how the curvatures of the surface model match different areas of the surfaces model against which it is compared. Alternatively and/or additionally, in some embodiments, the donor site surface models can be manipulated, e.g., rotated, to compare how those surfaces match the surface model of the defect region in different spatial orientations. The manipulation of the surface models may be performed using the rendering application which was used to generate the surface model or by using a separate application which can perform the manipulation using the rendered models. The results of these comparisons may be expressed using, e.g., a matching score or metric representative of how well the two surfaces matched at the particular positions and/or orientations. The level of matching may be based on the extent to which the curvatures and dimensions of the surfaces being compared fit each other, i.e., to what extent the two surfaces are congruent to each other. Such a determination may be performed by, e.g., minimizing the difference between the topologies represented by the two surface models (such as finding min(Σ_{x,y,z} V_{defect(x,y,z)}−V_{donor(x,y,z)})), where V represents topology vector values by minimizing the least-square error of the difference between the surface model representations of the donor and defect sites. In some exemplary embodiments, the optimal matching position orientation of the model surfaces compared may be performed visually by the operator of the system, e.g., a surgeon, who examines the surfaces compared to each other and selects one position/orientation which appears to result in the best match, or by a processing device. The procedure of matching the model surface of the defect region to model surfaces of potential donor sites can be repeated for other sites.

To compare the surface model of a defect region to one or more donor site surface models through, e.g., computations based on topological features of the surfaces, and the like, the operations may be facilitated by overlaying the surface models against each other. The overlaying operations may be achieved by using built-in overlaying functions available on the particular graphical rendering application being implemented. For example, when using SolidWorks, the application's alignment function may be used to position two or more surface model appearing in a view against each other. Alternatively and/or additionally, custom-made procedures for aligning and/or overlaying multiple surface models may be implemented for use with the particular rendering application or independently of the particular rendering application.

As described herein, the donor database 507 may include donor sites from which irregularly-shaped bone-cartilage grafts, e.g., non-cylindrical grafts, can be harvested. Thus, in situations where the defect region has an irregular shape and the optimal shape of the graft would be one that is substantially similar to the irregular shape of the defect region, a surface model of the irregularly-shaped defect, generated in the manner described herein, can be used to identify suitable donor sites from which irregularly-shaped grafts can be harvested. Specifically, the surface model of such an irregularly-shaped defect region, which includes small surface segments representative of dimensions and curvatures defining the irregular shape, can be compared against one or more donor sites stored in the database 507 with respect to which similar dimension and curvature information is maintained. As described herein, such a comparison may be performed by computing, e.g., a minimum of the difference (or the least-square error) between the surface features of the surface models of the defect and surface features of the surface models of candidate donor sites. In performing such comparisons, the donor surface models and/or the surface model of defect region may be re-positioned and have their orientations manipulated to enable comparing surface features of the defect region against sub-areas in a particular donor site surface model. In other words, the matching of a defect region, e.g., an irregularly-shaped defect, includes, in some exemplary embodiments, not only identifying a suitable donor site, but also identifying appropriate sub-areas and orientations at the donor site.

In some exemplary embodiments, after identifying an appropriate position where the model surface of the defect region matches (or reasonably matches) the model surface of the donor site, a cross-sectional tool to obtain cross-sections of each surface relative to the other may be used. Such a cross-sectional tool may be implemented on the application used to render the models, e.g., Pro/Engineer, SolidWorks 3D, and the like, or by using another application, e.g., a software implemented tool. The cross-sections of each surface may be overlapped to determine congruence of, e.g., surface textures, contours, and the like.

To identify suitable donor sites, comparisons of the surface model corresponding to the defect region to surface models from the donor database may be performed according to a hierarchy of matching criteria. Thus, identified suitable donor sites may be ranked to provide a hierarchy of suitable sites from which a user, e.g., a surgeon, may select one or more of the listed sites. Examples of matching criteria include the dimensions and/or topological attributes of the donor sites, the anatomical characteristics of the defect region and/or the donor sites, the compatibility of like and unlike joints relative to the defect region, the compatibility of like and unlike joints relative to other joints, the defect directionality, the area around the defect, and the like. In some embodiments, evaluation of the quality of a particular suitable site may be performed in a manner analogous to the matching level score described above, in which the extent of how well the surface of the defect region matches the surface of a potential donor site is determined and a representative “topographical matching” score is generated. Another example of a matching criterion is the impact of the harvesting bone-cartilage from a particular donor site will have on the well-being of the individual. Particularly, harvesting bone-cartilage from one particular anatomical location may affect the mobility of the patient (in that the bone-cartilage may be used, under some circumstances, during movement of the patient), while harvesting bone-cartilage from another anatomical location may have little or no impact on the mobility of the patient (in that the bone-cartilage is not utilized for mobility). Accordingly, another score, i.e., “impact” score, may be computed to represent the impact of harvesting bone-cartilage from a potential donor site. For example, various anatomical locations may be associated with predetermined impact values indicative of the impact harvesting bone-cartilage from the particular anatomical location would have on a patient's mobility or well-being. In some embodiments, a composite score which factors in the various scores derived for a particular anatomical location using the matching criteria may be determined. Such a composite score may be computed, in some embodiments, as a weighted average of the various computed criteria scores for the anatomical location.

Thus, and with reference again to FIG. 19, having identified suitable donor sites from which bone-cartilage can be harvested, and, in some embodiments, having ranked those sites, one or more of the identified sites can be selected by, e.g., a surgeon (604). Optionally, templates to harvest bone-cartilage and/or remove damaged bone-cartilage may be generated (605). Alternatively, the templates used may be selected from a repertoire of standard, pre-generated templates. Generating custom templates may be based, at least in part, on the received data corresponding to the defect region and/or on the data corresponding to the identified donor sites (and/or their associated surface model data) from which bone-cartilage graft(s) to repair the defect can be harvested. In some embodiments, generating custom templates can be performed using a 3D printer, such as the 3D printer 508 depicted in FIG. 17. The surgeon may then proceed to perform the harvesting procedures at the selected sites by utilizing the instruments, systems and methods described above (606).

The timing between scanning the defect region of the patient, requesting a bone-cartilage graft from, e.g., an allograft company, and obtaining the harvested bone-cartilage graft for implantation into the patient can vary significantly. For example, days, weeks or months can pass between performing an imaging scan of the defect region and obtaining a harvested bone-cartilage graft for implantation into the patient. During this time period, considerable changes to the defect region can occur, such as, e.g., increases of the defect region size, changes to the defect region topography, changes to the defect region shape, and the like. Thus, by the time the harvested bone-cartilage graft is received by the surgeon, it may not be fully compatible with the altered defect region. In addition, the data received with respect to the defect region may not be interpreted properly, thus resulting in a harvested bone-cartilage graft which is not compatible with the defect region. In such situations, the harvested bone-cartilage graft is generally disposed of by the surgeon, requiring the patient to wait a longer time period to receive an updated graft. This repetitious behavior creates a waste of graft materials and/or increases the damage incurred at the defect region by the patient.

As such, with reference to FIG. 21, a flowchart of an exemplary procedure 800 of graft cartilage management for identifying suitable donor sites for bone-cartilage grafts for repairing a defect in a joint of a patient is provided. Initially, a computing device, such as a computer or the controller processor 505 depicted in FIG. 17 accesses a donor database that includes information on each of a plurality of donor sites of like and/or unlike joints that may have been compiled and/or evolved from several sources of data (801). As discussed above, the database 507 of the controller processor 505 may have been populated with data downloaded, or otherwise retrieved, from remote locations which maintain data regarding potential donor sites. In some exemplary embodiments, the data may have been obtained by acquiring raw data, e.g., image data obtained using conventional imaging techniques, such as MRI imaging, CT imaging, ultrasound imaging, laser scans, and the like, from specimens having healthy cartilage of specific anatomical locations, e.g., joints which do not have defects or are otherwise non-injured. In some exemplary embodiments, the data may have been obtained through, e.g., topography capture using instruments with elongated pin members, such as trial member 100.

The procedure 800 further includes receiving a first data set relating to the defect region of the patient (802). As noted above, the defect region includes an area of a bone, a portion of which includes at least one of a missing and/or damaged cartilage. In some exemplary embodiments, the first data set relating to the defect region can be, e.g., imaging data of the defect region, imaging data of an area surrounding the defect region, data relating to the size of the defect region, data relating to the surface topography of the defect region and/or the area surrounding the defect region, data relating to the anatomical characteristics of the joint, and the like. The anatomical characteristics can be, e.g., the cartilage thickness, the cartilage density, the cartilage durability/resiliency, and the like. Such data may be acquired by the imaging and/or non-imaging techniques previously discussed. Surface models, e.g., 3D models, may also be generated for the defect region and/or the potential donor sites.

Once the first data has been received and/or the surface model has been generated, at least one compatible donor site from the donor database can be identified based on the first data relating to the defect region (803). The identification process can be performed by a processing device, e.g., the controller processor 505 illustrated in FIG. 17. Identifying the at least one donor site may include performing comparisons of the data representative of, for example, surface models of donor sites from the database to the first data relating to the defect region, anatomical characteristics of the donor sites from the database relative to the anatomical characteristics of the defect region, and/or to some derivative data thereof. In some exemplary embodiments, a prioritization of the identified donor sites may be performed to determine which donor site is the ideal match to be used for harvesting a bone-cartilage specimen (804). For example, if the identified donor sites include a plurality of unlike joints relative to the joint of the patient, a prioritization or ranking may be based on, e.g., the availability of alternative like or unlike joints, the compatibility of the identified unlike joint donor sites with other joints, and the like.

Rather than identifying a donor site for harvesting a bone-cartilage graft which precisely matches the first data received for the defect region, the exemplary procedure 800 involves harvesting a bone-cartilage specimen which is dimensioned greater than the defect in the joint of the patient. In particular, the bone-cartilage specimen can be selected partially based on the first data such that a bone-cartilage graft matching at least some of the first data, e.g., the defect area topography, the cartilage anatomical characteristics, and the like, can be subsequently harvested from the bone-cartilage specimen. The donor site for the bone-cartilage specimen is thereby identified without trying to exactly match the defect region with the data for the donor site. Since the size of the defect region can change between scanning the defect region and obtaining a bone-cartilage graft for implantation, the dimensions of the bone-cartilage specimen can be greater than the defect region to permit a surgeon to subsequently customize the size of the graft to be implanted on site, e.g., in the operating room prior to implantation. In some exemplary embodiments, imaging can be used to reduce the size of the donor site needed for harvesting the bone-cartilage specimen while knowing that within the bone-cartilage specimen is an area that presents a substantial match for the first data of the defect region. For example, rather than sending a full joint for harvesting of a bone-cartilage defect, the joint may be reduced into, e.g., halves, thirds, and the like, based on the selection of the bone-cartilage specimen. This increases the amount of donor joints available for harvesting other bone-cartilage specimens. Based on these comparisons (and, optionally, the prioritization), at least one suitable donor site can be selected by, e.g., a surgeon, for harvesting the bone-cartilage specimen (805).

Templates for harvesting the bone-cartilage specimen may be generated. Alternatively, templates used may be selected from a repertoire of standard, pre-generated templates. Generating custom templates may be based, at least in part, on the received data corresponding to the defect region and/or on the data corresponding to the identified donor sites from which bone-cartilage graft(s) to repair the defect region can be harvested. In some exemplary embodiments, generating custom templates can be performed using a 3D printer, such as the 3D printer 508 depicted in FIG. 17.

With continued reference to FIG. 21, the bone-cartilage specimen which is dimensioned greater than the defect region of the patient can be harvested from the selected donor site with, e.g., the harvesting instruments discussed herein (806). The bone-cartilage specimen can then be delivered to the surgeon performing the surgery for repairing the defect region of the patient (807). As described above, the bone-cartilage specimen defines an area greater than the defect region, i.e., greater than the first data received. As such, when the bone-cartilage specimen is received by the surgeon, a second data set relating to the defect region as it exists at the time of surgery can be collected to determine if the defect region has changed since the first data set has been collected (808). The second data set can be, e.g., imaging data of the defect region, imaging data of an area surrounding the defect region, data relating to the size of the defect region, data relating to the surface topography of the defect region and/or the area surrounding the defect region, data relating to the anatomical characteristics of the joint, and the like. In some exemplary embodiments, the second data set can be collected by utilizing the exemplary trial member 100 of FIG. 1. In particular, the elongated rod members 101 of the trial member 100 can be utilized to capture the surface topography of the defect region. The captured surface topography of the defect region can then be utilized to identify the complementary surface topography on the bone-cartilage specimen.

Once a complementary surface topography on the bone-cartilage specimen has been identified, a bone-cartilage graft can be harvested by the surgeon based on the second data relating to the defect region at the time of surgery (809). In particular, the bone-cartilage graft can be harvested to substantially match the defect region at the time of surgery, e.g., the defect region topography, the topography surrounding the defect region, the defect region size, and the like. Thus, rather than harvesting a bone-cartilage graft based on data representing the old configuration and/or dimensions of the defect region, harvesting of the bone-cartilage graft at the time of surgery ensures an ideal fit of the graft in the defect region by matching the graft configuration and/or dimensions to the defect region as it exists at the time of surgery. In some exemplary embodiments, templates for removing damaged bone-cartilage from the defect region of the patient may be generated by procedures substantially similar to those used for generating the templates for harvesting the bone-cartilage specimen. The defect region can thereby be removed and the recently-harvested bone-cartilage graft can be implanted to repair the defect region in the joint of the patient.

Turning now to FIG. 22, a diagram of an exemplary surface mapping system 850 is provided for acquiring data regarding cartilage-bone anatomies and/or defect region anatomies and to enable identification of suitable donor sites to harvest cartilage from or to enable identification of suitable defect regions for receiving a bone-cartilage graft to repair defects in a patient's bone. In particular, rather than maintaining a database of suitable donor sites and receiving information related to a defect area for matching the defect area to a suitable donor site, as will be described below, the exemplary system 850 can include a database which also includes (or in the alternative includes) information, e.g., MRI data, surface topography data, joint anatomical characteristics data, and the like, on a plurality of defect regions of various patients. System 850 can further receive data for an incoming donor site and can match the donor site to a suitable defect region to be repaired. System 850 will be described below with respect to a database including defect region information and receiving data on a donor site to determine the suitable defect region to be matched for receiving a graft harvested from the donor site. However, it should be understood that system 850 may be combined with system 500 described above to perform the matching of defect regions to donor sites and vice versa.

System 850 generally includes an imaging apparatus 851 for capturing image and/or non-image data of an area on a donor's body, e.g., a donor's foot 852, which includes a suitable bone-cartilage donor site. The imaging apparatus 851 includes, e.g., one or more of an MRI system, a computed tomography apparatus configured to generated three-dimensional images from a series of two-dimensional images (e.g., X-Ray) taken around a single axis of rotation, a Medical sonography (ultrasound) imaging device, and any other suitable imaging device to acquire data representative of anatomical structures in a donor's body. In some embodiments, a non-imaging apparatus may also be used to capture data regarding, e.g., anatomical characteristics of the donor site, and the like. In some exemplary embodiments, the data relating to the donor site of the donor may have been acquired at an earlier and/or at some location other than where the system 850 is located, in which case, the data may be received at the system 850 from some remote location which can electronically communicate, e.g., via one or more types of communication networks such as a network 853, including the Internet, a telephony network, and the like, the data relating to the donor site of the donor.

The imaging apparatus 851 acquires one or more images of the donor site which is suitable for bone-cartilage graft harvesting, e.g., the talus surface at the foot 852. It should be understood that the donor site may be located anywhere in the body. In some exemplary embodiments, system 850 also includes a signal processing unit 854 connected to the imaging apparatus 851. The processing unit 854 receives the signals communicated from the imaging apparatus 851 and performs signal processing and/or enhancement operations, e.g., amplification, filtering, and the like. For example, the processing unit 854 may be configured to perform noise reduction to remove noisy artifacts from acquired image data. Other types of processing may include image processing operations to transform the image data into resultant data which can be more easily manipulated for the purpose of identifying a suitable defect region. For example, the acquired data can be processed to generate a surface model corresponding to the donor site, transform spatial representations into another domain, e.g., the frequency domain, which is more conductive for various types of processing, and the like.

The processed data can subsequently be communicated to the controller processor 855. The controller processor 855 includes a storage device 856 to store the data (processed and/or raw acquired data) relating to the donor site, and to store a defect region database 857 which includes information on each of a plurality of defect regions seeking a compatible donor site for harvesting a bone-cartilage graft. It should be understood that if the system 850 is utilized in combination with system 500 described above, the controller processor may include, e.g., separate storage devices 856 for storing data relating to the donor site and the defect region, one storage device 856 for storing data relating to both the donor site and the defect region, separate databases 857 for storing information on a plurality of defect regions and donor sites, one database 857 for storing information on both the plurality of defect regions and donor sites, and the like. As discussed above, the database 857 may be constructed based on data acquired from multiple sources and/or multiple patients. The acquired data may be used to develop and/or expand the database 857 and enhance the sensitivity and specificity of the system 850. Typically, the data stored on the database 857 pertains to defect regions, i.e., injured joints of patients, thus enabling identification of a suitable defect region to be repaired with a bone-cartilage graft harvested from the incoming donor site. The controller processor 855 can thus be configured to receive a first data relating to a donor site and identify, based on the received first data, at least one defect region from the defect region database 857 which can be repaired by a graft harvested from the incoming donor site. In some exemplary embodiments, as described above, the controller processor 855 and/or the database 857 can include correlation information indicating which joints of the body can be used for harvesting of bone-cartilage grafts to be implanted in different unlike joints of the body based on anatomical characteristics of the joints, e.g., cartilage thickness, cartilage density, cartilage resiliency/durability, and the like. Thus, the incoming donor site can be matched with compatible defect regions in like and unlike joints.

In some embodiments, the storage device 856 hosting the defect region database 857, or another storage device hosting the database 857, may be located at one or more remote locations which may be accessed by multiple systems, such as system 850. Thus, such a remote device may serve as a central data repository on which data pertaining to defect regions may be stored. A user locally interacting with system 850 may therefore access remotely, via a network 853, a database such as the database 857 to retrieve data as required. For example, and as will be described in greater detail below, data pertaining to potential defect regions which is compared to data relating to a donor site can be retrieved from a remote location. Optionally, a 3D printer 858 may be locally or remotely interconnected to the controller processor 855. Such a 3D printer 858 may be used to create 3D custom templates corresponding to any identified defect region and/or to the donor site. Similar to the controller processor 505 described above, the controller processor 855 may be configured to perform learning functions.

With reference now to FIG. 23, an exemplary procedure 900 for defect region management is provided for identifying suitable defect regions for receiving a bone-cartilage graft harvested from a donor site to repair the defect region of the patient. Initially, a computing device, such as a computer or the controller processor 855 depicted in FIG. 22, accesses the defect region database 857 which includes information on each of a plurality of defect regions which are in need of a bone-cartilage graft that may have been compiled and/or evolved from several sources of data (901). In some exemplary embodiments, the data may have been obtained by acquiring raw data, e.g., image data obtained using conventional imaging techniques, such as MRI imaging, CT imaging, ultrasound imaging, laser scans, and the like. For example, in some embodiments, data acquired may be representative of the topology, health and other physiological attributes, such as gender, age, race, activity index, BM1, V02, cartilage thickness, bone density, and the like, of the defect regions and/or the area surrounding the defect regions.

The procedure 900 further includes receiving a first data set relating to a donor site from which a bone-cartilage graft may be harvested (902). In some embodiments, the data relating to the donor site may be acquired by imaging techniques and/or non-imaging techniques. Surface models of the donor site may also be constructed, including data regarding the topology of the area, as well as other information descriptive of the area, e.g., bone thickness, bone density, bone resiliency/durability, and the like. Once the first data has been received and/or the data was used to generated a surface model to be used in identifying a suitable defect region from the defect region database 857, at least one defect region from the defect region database 857 can be identified based on the first data relating to the donor site (903). Identifying the at least one defect region may include performing comparisons of the data representative of, for example, surface models of the defect regions from the database 857 to the first data relating to the donor site, or to some derivative data thereof. In some exemplary embodiments, a prioritization of the identified defect regions may be performed to determine which defect region is the ideal match to receive a bone-cartilage graft harvested from the donor site (904). For example, if some of the identified defect regions can receive a graft from alternative donor sites, while other identified defect regions cannot receive a compatible graft from alternative donor sites, a prioritization or ranking may be performed to determine which defect region should receive a bone-cartilage graft harvested from the donor site. In some exemplary embodiments, the prioritization can be based on, e.g., the length of time a patient has waited to receive a graft implantation, the complexity involved in locating a graft which is compatible to the bone-cartilage of the patient, and the like. Thus, having identified suitable defect regions from and, in some embodiments, having prioritized or ranked the defect regions, one or more of the identified defect regions can be selected by, e.g., a surgeon (905).

In some exemplary embodiments, when one or more of the identified defect regions have been selected, templates to harvest bone-cartilage and/or remove damaged bone-cartilage may be generated. For example, a template can be generated to remove the damaged bone-cartilage from the defect region and a complementary template can be generated to harvest the bone-cartilage graft for repairing the defect region from the donor site. The surgeon can then proceed to perform the harvesting procedure at the donor site based on the data relating to the selected defect region by utilizing the instruments, systems and methods described above (906). Once the bone-cartilage graft has been harvested from the donor site, the bone-cartilage graft can be delivered to a surgeon for implantation of the graft into the patient to repair the defect region, i.e., the joint of the patient requiring the graft (907).

In some exemplary embodiments, when one or more of the identified defect regions have been selected, a template to harvest bone-cartilage from the donor site can be generated partially based on the data relating to the selected defect region. In particular, similar to the process 800 described above, the bone-cartilage graft can be harvested to be dimensioned greater than the data relating to selected defect region to receive the graft, while matching certain anatomical characteristics for the defect region, e.g., a surface topography, the cartilage density, the cartilage resiliency/durability, the cartilage thickness, and the like (908). However, the bone-cartilage graft can be harvested such that within the bone-cartilage graft exists an area which can be harvested and utilized to repair the defect region on site, e.g., in the operating room prior to the operation, during the operation, and the like. Thus, the harvested bone-cartilage graft can be delivered to the surgeon for further customizing with respect to the defect region to be repaired (909).

When the bone-cartilage graft is received by the surgeon, a second data set relating to the selected defect region as it exists at the time of surgery can be collected to determine if the defect region has changed since the initial imaging and/or non-imaging data was collected for the defect region database 857 (910). The second data set can be, e.g., imaging data of the defect region, imaging data of an area surrounding the defect region, data relating to the size of the defect region, data relating to the surface topography of the defect region and/or the area surrounding the defect region, data relating to the anatomical characteristics of the joint, and the like. In some exemplary embodiments, the second data set can be collected by utilizing the exemplary trial member 100 of FIG. 1. In particular, the elongated rod members 101 of the trial member 100 can be utilized to capture the surface topography of the defect region. The captured surface topography of the defect region can then be utilized to identify the complementary surface topography on the bone-cartilage graft.

Once a complementary surface topography on the bone-cartilage graft has been identified, a custom bone-cartilage graft can be harvested by the surgeon based on the second data relating to the defect region at the time of surgery (911). In particular, the custom bone-cartilage graft can be harvested to substantially match the defect region at the time of surgery, e.g., the defect region topography, the topography surrounding the defect region, the defect region size, and the like. Thus, rather than harvesting a bone-cartilage graft based on data representing the old configuration and/or dimensions of the defect region which was included in the defect region database 857, harvesting of the custom bone-cartilage graft at the time of surgery ensures an ideal fit of the graft in the defect region by matching the graft configuration and/or dimensions to the defect region as it exists at the time of surgery. The defect region can thereby be removed, e.g., with templates, and the recently-harvested custom bone-cartilage graft can be implanted to repair the defect in the joint of the patient.

In some exemplary embodiments, the procedure 900 can be advantageously used for instances where one or more patients with defect regions are awaiting a compatible donor site for harvesting an implant to repair the defect region. For example, although a donor database includes a plurality of donor sites available for harvesting bone-cartilage grafts, the available donor sites may not be compatible with, e.g., the shape of the defect region, the size of the defect region, the anatomical characteristics of the defect region, the surface topography surrounding the defect region, the surface topography of the defect region, and the like, for all patients. As a further example, an insufficient amount of donor sites may be available, requiring some patients to wait until a new compatible donor site becomes available. Thus, when a new donor site, e.g., allograft tissue, and the like, becomes available, imaging and/or non-imaging techniques discussed herein can be used to scan and compare the new donor site to the data relating to defect regions requiring a bone-cartilage graft, e.g., MRI data, generated surface models, surface topography captured/measured by instruments discussed herein, and the like. In particular, the exemplary system can use the data relating to the new donor site to scan the database, e.g., a library, of data relating to defect regions of patients and can determine the best match. If a match between the new donor site and a defect region is found, the procedures discussed herein can be used to harvest the appropriate bone-cartilage graft for repairing the defect region. Patients requiring a bone-cartilage graft to repair a defect region can thereby be efficiently matched as soon as compatible donor sites become available.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A method of graft cartilage management for repairing a defect in a joint of a patient, the method comprising:
providing a donor database comprising information on a plurality of donor sites;
receiving first data relating to the defect of the joint of the patient; and
identifying, based on the first data, at least one donor site from the donor database for harvesting a graft specimen dimensioned greater than the defect in the joint of the patient.

2. The method according to claim 1, wherein the first data comprises at least one of imaging data of the defect, imaging data of an area surrounding the defect, a size of the defect, a surface topography of the defect, and a joint anatomical characteristic.

3. The method according to claim 1, wherein the joint anatomical characteristic comprises at least one of a cartilage density, a cartilage thickness, and a cartilage resiliency.

4. The method according to claim 1, further comprising prioritizing the identified at least one donor site.

5. The method according to claim 4, wherein prioritizing the identified at least one donor site is based on at least one of a donor site availability and a donor site compatibility.

6. The method according to claim 1, wherein the identified at least one donor site comprises a like joint with respect to the joint of the patient.

7. The method according to claim 1, wherein the identified at least one donor site comprises an unlike joint with respect to the joint of the patient.

8. The method according to claim 1, further comprising harvesting the graft specimen from the at least one donor site and delivering the graft specimen to a surgeon.

9. The method according to claim 8, further comprising collecting second data relating to the defect of the joint of the patient.

10. The method according to claim 9, wherein the second data comprises at least one of imaging data of the defect, imaging data of an area surrounding the defect, a size of the defect, a surface topography of the defect, and a joint anatomical characteristic.

11. The method according to claim 9, further comprising harvesting a graft from the graft specimen based on the second data relating to the defect of the joint of the patient.

12. A graft cartilage management system for repairing a defect in a joint of a patient, the system comprising:
a donor database comprising information on a plurality of donor sites; and
a processing device configured to (i) access the donor database, (ii) receive first data relating to the defect of the joint of the patient, and (iii) identify, based on the first data, at least one donor site from the donor database for harvesting a graft specimen dimensioned greater than the defect in the joint of the patient.

13. The system according to claim 12, further comprising an apparatus to capture the first data relating to the defect of the joint of the patient.

14. The system according to claim 13, wherein the first data comprises at least one of imaging data of the defect, imaging data of an area surrounding the defect, a defect size, a defect surface topography, and a joint anatomical characteristic.

15. The system according to claim 12, further comprising a harvesting device for harvesting the graft specimen from the at least one donor site.

16. The system according to claim 12, further comprising an apparatus to capture second data relating to the defect of the joint of the patient.

17. The system according to claim 16, further comprising a harvesting device for harvesting a graft from the graft specimen based on the second data relating to the defect of the joint of the patient.

18. A method of graft cartilage management for repairing a defect in a joint of a patient, the method comprising:
providing a donor database comprising information on a plurality of donor sites, wherein the plurality of donor sites include like joint donor sites and unlike joint donor sites relative to the joint of the patient;
receiving first data relating to the defect of the joint of the patient; and
identifying, based on the first data, at least one unlike joint donor site from the donor database for harvesting a graft to repair the defect in the joint of the patient.

19. The method according to claim 18, wherein the at least one unlike joint donor site is compatible with the joint of the patient.

20. The method according to claim 19, wherein the at least one unlike joint donor site is compatible with the joint of the patient based on at least one of imaging data of the defect, imaging data of an area surrounding the defect, a size of the defect, a surface topography of the defect, and a joint anatomical characteristic.

21. The method according to claim 20, wherein the joint anatomical characteristic comprises at least one of a cartilage density, a cartilage thickness, and a cartilage resiliency.

22. A graft cartilage management system for repairing a defect in a joint of a patient, the system comprising:
a donor database comprising information on a plurality of donor sites, wherein the plurality of donor sites include like joint donor sites and unlike joint donor sites relative to the joint of the patient; and
a processing device configured to (i) access the donor database, (ii) receive first data relating to the defect of the joint of the patient, and (iii) identify, based on the first data, at least one unlike joint donor site from the donor database for harvesting a graft to repair the defect in the joint of the patient.

23. A method of defect region management for repairing a defect in a joint of a patient, the method comprising:
providing a database comprising information on a plurality of joint defects;
receiving first data relating to a donor site; and
identifying, based on the first data, at least one joint defect from the database for repair by a graft harvested from the donor site.

24. The method according to claim 23, wherein the first data comprises at least one of imaging data of the donor site, a size of the donor site, a surface topography of the donor site, and a joint anatomical characteristic.

25. The method according to claim 23, further comprising prioritizing the identified at least one joint defect.

26. The method according to claim 23, further comprising harvesting the graft from the donor site.

27. The method according to claim 23, wherein the graft is dimensioned greater than the identified at least one joint defect.

28. The method according to claim 27, further comprising collecting second data relating to the identified at least one joint defect and harvesting a custom graft based on the second data relating to the identified at least one joint defect.

29. A defect region management system for repairing a defect in a joint of a patient, the system comprising:
a database comprising information on a plurality of joint defects; and
a processing device configured to (i) access the database, (ii) receive first data relating to a donor site, and (iii) identify, based on the first data, at least one joint defect from the database for repair by a graft harvested from the donor site.
